(19) United States
(12) Patent Application Publication
(10) Pub. No.: US 2026/0253695 A1
(43) Pub. Date: Aug. 27, 2026

(54) MEDICAL TEST RESULT REPORTING USING BARCODES

(71) Applicant: ADL DIAGNOSTICS, INC., Redmong, WA (US)

(72) Inventor: Robert G. Atkinson, Woodinville, WA (US)

(21) Appl. No.: 19/156,923

(22) PCT Filed: Feb. 8, 2024
(86) PCT No.: PCT/US2024/015027

§ 371 Date: Aug. 15, 2025

Related U.S. Application Data
(60) Provisional application No. 63/484,447, filed on Feb. 10, 2023.

(51) Int. Cl. G16H 15/00; G06K 19/06

(52) U.S. Cl. CPC G16H 15/00; G06K 19/06028

(57) ABSTRACT

Systems and methods for detecting an analyte in a sample are disclosed. A system may be operable to amplify an analyte and measure a signal associated with a quantity of the analyte, according to an embodiment. The system may be configured to display a dynamically generated barcode for communicating test results to a patient, a medical professional, or to an agency for analyzing pathogen transmission within a population. The barcode may be a dynamically generated matrix code such as QR code (e.g., a QR code presented via an LCD or other suitable display that can be customized based on, e.g., test results). The system may include a housing, which includes a well, a receiver, a light source, and a light sensor. Further, the housing may include a processor, an interface, and a display configured to display various test-related information, such as, for example, the dynamically generated barcode.

### CROSS-REFERENCE TO RELATED APPLICATIONS

This application claims the benefit of, and priority to, U.S. Provisional Application No. 63/484,447 filed on Feb. 10, 2023, the content of which is expressly incorporated herein by reference in its entirety for any and all non-limiting purposes.

### FIELD

Embodiments described herein generally relate to systems and methods for reporting diagnostic test results using barcodes. Various embodiments described herein are suitable for reporting diagnostic tests, for example, determining whether a sample taken from a patient contains a pathogen such as a severe acute respiratory syndrome coronavirus 2 (SARS-CoV-2), or a flu virus.

### BACKGROUND

A number of diagnostic and analytic techniques have been developed to detect the presence of proteins, DNA, or other suitable biomarkers, for example, those associated with SARS-CoV-2. Many such techniques are designed to amplify a target analyte for a predetermined period of time and then determine whether a quantity of the target analyte is detectable and/or exceeds a predetermined threshold that indicates a “positive” result. In many situations it may be desirable to report the results of the analysis, for example, to a remote and/or central authority. For example, the information indicating a “positive” or a “negative” result can be delivered to a medical professional, to a patient, or to an agency for determining transmission levels of the pathogen within a population. Presently, the test results may be entered manually via a computer interface. Alternatively, the diagnostic test equipment may include network connectivity for reporting such results automatically. These techniques, however, may be unsuitable in “pop-up” style clinics or testing sites where networking infrastructure may be inadequate to support large numbers of connected computers or diagnostic devices. Thus, a need exists for systems and methods of reporting diagnostic test results and collecting information about these test results.

### SUMMARY

Systems and methods described herein are well suited for “rapid” testing and test result reporting, optionally using nearly ubiquitous user smartphones without specialized apps or configurations, which can significantly contribute to curbing the spread of a pathogen such as COVID-19.

### BRIEF DESCRIPTION OF THE FIGURES

FIG. 1 shows an example of a diagnostic testing device for determining a presence of one or more of different types of pathogens in a test sample and reporting test results via a barcode according to an embodiment.

FIG. 2 shows an example implementation of the device shown in FIG. 1.

FIG. 3A shows an example representation of data records stored on a server, where each data record is associated with a corresponding device and is used for authenticating the device according to an embodiment.

FIG. 3B shows example data communications between various entities that can be established with a test server according to an embodiment.

FIGS. 4A and 4B show examples of QR codes.

FIG. 5 shows example plots of data signals corresponding to positive and negative test results according to an embodiment.

FIGS. 6A and 6B shows examples of messages that can be displayed by a diagnostic testing device according to an embodiment.

FIG. 7 shows an example of a webpage provided after scanning a device-related barcode according to an embodiment.

FIG. 8A is an example of a webpage provided after scanning a test result-related barcode according to an embodiment.

FIG. 8B shows an example of an output of a test result on a webpage provided after scanning a test result-related barcode according to an embodiment.

FIG. 9 shows an example of a process for performing a diagnostic test, encoding data, as well as forming a barcode from the encoded data according to an embodiment.

FIG. 10 shows an example of a process for providing a website based on data of a barcode according to an embodiment.

### DEFINITIONS

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

The term “including” is used to mean “including but not limited to.” “Including” and “including but not limited to” are used interchangeably.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, the term “sample” refers to a composition that contains an analyte or analytes. A sample can be heterogeneous, containing a variety of components or homogenous, containing one component. In some instances, a sample can be naturally occurring, a biological material, and/or a man-made material. Furthermore, a sample can be in a native or denatured form.

In certain embodiments, the sample is a biological sample. In some instances, a sample can be a single cell (or contents of a single cell) or multiple cells (or contents of multiple cells), a saliva sample, a mucous sample, a blood sample, a tissue sample, a skin sample, a urine sample, a water sample, and/or a soil sample. In some instances, a sample can be from a living organism, such as a eukaryote, prokaryote, mammal, human, yeast, and/or bacterium or the sample can be from a virus. In some embodiments, a sample can be a food product or a beverage product. In some embodiments, a sample can be a swab of a surface, e.g., a swab of a food preparation surface or a container. Biological samples include, but are not limited to, tissues, cells and biological fluids obtained from a subject. For example, biological samples include, but are not limited to, blood and a fraction or component of blood including blood serum, blood plasma, or lymph, saliva, nasal fluid, etc. In certain embodiments, the biological sample is a blood sample, a serum sample, a saliva sample, a mucous sample, a tissue sample, a skin sample, or a urine sample. In one embodiment, the biological sample contains virus or protein molecules from the test subject. The biological sample may be a peripheral blood leukocyte sample isolated by conventional means from a subject. In certain embodiments, the biological sample is selected from the group consisting of: serum, blood, salivary secretions (e.g., saliva), lacrimal secretions (e.g., tears), respiratory secretions (e.g., mucus), nasal fluid, a nasal swab, an oral swab, a mucous sample, and intestinal secretions (e.g., mucus).

As used herein, the term “analyte” refers to any molecule or compound to be detected as described herein. Suitable analytes can include but are not limited to, small chemical molecules and/or biomolecules, such as, for example, environmental molecules, clinical molecules, chemicals, and pollutants. More specifically, such chemical molecules and/or biomolecules can include but are not limited to pesticides, insecticides, toxins, therapeutic and/or abused drugs, hormones, antibiotics, antibodies, organic materials, proteins (e.g., enzymes, immunoglobulins, and/or glycoproteins), nucleic acids (e.g., DNA and/or RNA), lipids, lectins, carbohydrates, whole cells (e.g., prokaryotic cells such as pathogenic bacteria and/or eukaryotic cells such as mammalian tumor cells), viruses, spores, polysaccharides, glycoproteins, metabolites, cofactors, nucleotides, polynucleotides, transition state analogs, inhibitors, nutrients, electrolytes, growth factors and other biomolecules and/or non-biomolecules, as well as fragments and combinations thereof. Some analytes described herein can be proteins such as enzymes, drugs, cells, antibodies, antigens, cellular membrane antigens, and/or receptors or their ligands (e.g., neural receptors or their ligands, hormonal receptors or their ligands, nutrient receptors or their ligands, and/or cell surface receptors or their ligands). In particular embodiments, an analyte is an infectious or pathological agent, such as, e.g., a bacterium, virus, yeast, or fungus.

As used herein, the term “protein” refers to proteins, polypeptides, oligopeptides, peptides, and analogs, including proteins containing non-naturally occurring amino acids and amino acid analogs, and peptidomimetic structures. The term “protein” also refers to proteins, polypeptides, oligopeptides, peptides, and analogs.

### DETAILED DESCRIPTION

Epidemics, pandemics, and other periods of widespread infection across a population may prompt health agencies, governmental agencies, and other types of agencies and organizations to monitor rates of infection. Diagnostic testing means may be available for rapid, high-volume testing across a geographically distributed population. In order to provide accurate information about the rates of infection and make informed recommendations or decisions based on such information, an entity must be confident that it has received authentic and reliable reports of testing results. As such, collecting and compiling test results from such geographically distributed testing sites raises technical considerations and technical challenges. For example, a communication channel is needed to carry information with sufficient markers of authenticity and reliability from the diagnostic testing devices to the entity collecting and compiling the test results.

Publicly available communication infrastructures such as the global Internet may be used to report and receive test results. Enabling diagnostic testing devices to provide results via electronic communication networks such as the Internet, however, can increase the costs and complexity of the design of such devices due to the additional hardware (e.g., wired and wireless interfaces) and programming (e.g., network protocols) included. Even if such additional hardware and programming were provided, other technical challenges may render wired or wireless communication means unsuitable to report test results in some scenarios. For example, large groups of diagnostic testing devices may be deployed at a common site for high-volume, high-throughput testing of patients. Using wired technologies (e.g., Ethernet, etc.) may not be desirable or feasible given the lack of or limits on available access points to a communication network. Using wireless technologies (e.g., cellular, Wi-Fi, Bluetooth, etc.) may not be desirable due to wireless interference caused by the devices being in close proximity with each other. Other technical requirements may result, in some scenarios, in certain wireless technologies being an undesirable solution for reporting test results from diagnostic testing devices. For example, some short-range communication standards, such as Bluetooth, may require successfully completing a pairing process before information can be exchanged between devices which introduces added complexity to the process of reporting test results (e.g., if a pairing process needs to be performed for each report transmission or if a paired state needs to be maintained for multiple report transmissions).

In addition, when using publicly available communication channels like the Internet to report test results, a mechanism for establishing that reported test results are authentic is needed. In other words, a mechanism is needed to differentiate between authentic results and noise (e.g., fake reports, garbled reports, and the like). Moreover, confidence in the underlying reports being provided may depend on knowing that a diagnostic testing device is operating correctly (e.g., not providing false positives and/or false negatives). As such, confidence in the reliability of the data may be maintained by including with the reported test results information indicating the operating status of the device (e.g., information indicting the “health” of the device). Such information may be helpful in determining whether a diagnostic testing device is operating properly and, in turn, the reliability of any test results received from that device. Providing sufficient markers of authenticity and reliability, however, is not trivial in all scenarios. Constraints associated with the diagnostic testing means may impose technical challenges on providing test results in a manner that enables an entity that receives the reported test results to identify and compile valid test reports from invalid test reports. As an example, a given form factor (e.g., a preferred for factor or a required form factor) might result in a diagnostic testing device with a relatively small footprint, limited hardware, and/or limited computational abilities. As such, equipping a diagnostic testing device to use existing technological measures, such as digital signatures or public key infrastructures, for enabling authentication of reported test results might not be desirable or feasible given the form factor, hardware, and/or computational constraints that are preferred or required. Furthermore, including additional information relating to the operating status of the diagnostic testing device increases the size of the payload to report. Therefore, there is a need for a mechanism that can efficiently report test results from a population of distributed testing stations with sufficient markers of authenticity and reliability.

In view of the technical challenges described above, techniques are described herein for a visual means of providing test results from a population of geographically distributed diagnostic testing devices to remotely located entities that collect and compile those test results. The techniques described herein leverage the existing technological capabilities of computing devices that facilitate the delivery of the test results from the diagnostic testing devices to those entities. The visual means for conveying the test results include a dynamically generated barcode, such as a two-dimensional (2D) barcode or matrix code, that is configured to, when scanned by a computing device, cause the computing device to deliver, among other things, the test results to a remotely located entity. The configuration of the dynamically generated barcodes is such that the inherent capabilities of the computing devices used to scan and process the barcodes are leveraged to facilitate the delivery of the test results and additional information encoded in the barcodes without any special programming being required by those computing devices.

Using a visual means to report test results with sufficient markers of authenticity, however, raises additional technical challenges. As noted above, the form factor of a diagnostic testing device may constrain the ability of the device to output visual information. For example, a screen display may be limited in size constraining the amount of information that may be presented at any given time. As such, one technical challenge arising from using a visual means to provide test results with sufficient markers of authenticity and reliability arises from the maximum size of a barcode (e.g., a matrix barcode) that can be presented on the screen display. In other words, the amount of information that can be stored in a barcode may depend on the size of the barcode used. The available storage space in a barcode may also depend on the level of error correction employed. Lower levels of error correction may allow for larger payloads but increase the possibility of errors during scanning and/or decoding. Higher levels of error correction may reduce the possibility of errors but also reduce the size of the payload due to more of the available storage space being used for error correction information. Furthermore, a screen display may need to provide a sufficient resolution (e.g., a minimum number of pixels per barcode unit) in order to successfully and properly read a barcode. For example, some matrix barcodes (e.g., QR codes) are comprised of a grid of black and white squares (barcode units). A sufficient resolution may include a 4×4 grid of pixels for each barcode unit/module (e.g., black or white square). A display screen of n×n (or n×m) pixels thus limits the size of any barcode presented. These technical considerations demonstrate tradeoffs between the amount of information that can be stored in a barcode (e.g., a matrix code) and the display screen size, the number of display screen pixels used per barcode unit, and the level of error correction employed. These challenges may be exacerbated where a barcode shares a screen display with other information (e.g., instructions, messages, control options, and the like). Such additional information may further limit the size of a barcode for a screen display of a given size and thus the amount of information that may be stored in that barcode.

Techniques described herein address the technical challenges described above to provide a dynamically generated barcode (e.g., a matrix code) with sufficient markers of authenticity and reliability and with sufficient resolution and error correction to minimize errors in the transmission and decoding. The techniques described below may be employed to maximize the amount of information that may be conveyed through a visual means given constraints or limitations imposed by the diagnostic testing device that presents the test results using a visual means. As described below, such techniques include the structure of the payload with the information to report, accompanying information used to authenticate the reported information, the encoding scheme used to encode such information, and the mechanism used to trigger a computing device to facilitate delivery of such information.

Techniques described herein are particularly suitable for efficiently communicating results of a diagnostic test for determining if a patient has a viral or bacterial infection (e.g., COVID-19, Flu, Respiratory Syncytial Virus (RSV), SARS, and the like) to a medical service provider, a patient, and/or a government agency for determining pathogen transmission levels in a population. As various tests are becoming available (e.g., PCR, loop-mediated isothermal amplification, antigen test, such as rapid antigen test, and the like), for example, for diagnosing a COVID-19 infection, it is desirable to disseminate information about test results to various agencies (e.g., government agencies, medical agencies, patients, patient relatives, etc.) for determining various safety protocols and approaches for reducing pathogen transmission in the population. However, due to privacy concerns, in many instances, test providers and government agencies may not communicate personal patient-related information, such as patient's name, address, age, phone, accompanying medical conditions, and the like. Therefore, the present disclosure describes systems and methods for performing diagnostic tests using a diagnostic test device, dynamically generating a barcode, and displaying the dynamically generated barcode to test service providers. Further, the present disclosure describes providing the test results to a patient and/or to a government or a health agency (e.g., if required by law to report test results). Additionally, test results may be provided with markers of authenticity thereby preventing a third party from altering the test results or submitting fake test results. Additionally, test results may be provided only to authorized entities in some cases.

In various embodiments described herein, the test results are performed by a Fluorescence of Loop Primer Upon Self Dequenching Loop-Mediated Isothermal Amplification (FLOS-LAMP) device. The device may be configured to determine the test results, encode the test results, communicate the test results via a network, and/or provide device authentication information when communicating the test results. Further, the device is configured to provide the test results in a form of a barcode (e.g., a matrix barcode such as a QR code), which, when scanned by a scanning device (e.g., a smartphone) allows a user (or a test service provider) to submit the test results to a remotely located entity such as, e.g., a “test server” configured to store the test results in a data store (e.g., one or more databases) associated with the user, the diagnostic testing device that performed the test, and/or a particular testing facility. In some cases, the “test server” may be configured to disseminate the information related to the test results to government and/or health organizations (e.g., if required to do so by law). The “test server” may also be configured to generate (e.g., automatically) documentation indicating the results of the test and provide such documentation to the patient as evidence of a positive or negative result. Such documentation may be provided to the patient using electronic means such as email or text message and/or physical means such as hardcopy documentation sent via post. Reports of diagnostic testing results may also be provided to other health-related systems, e.g., electronic health record (EHR) systems for updating EHRs.

In various embodiments, a test server may be configured to receive and accept test results only from known testing devices. For example, the payload of a report may include the test results along with a serial number of the diagnostic testing device that performed the test. The test server may compare the transmitted serial number to a database of serial numbers corresponding to various diagnostic testing devices and, if the transmitted serial number matches one of the serial numbers in the database, the test server may accept the test results and process the test results as described herein. For example, the test server may store the test results a provide a web portal (e.g., via website, a webpage, and the like) based on the received test results, the test server may disseminate the test results to the government and/or health organizations, and the like. In some instances, the test server may not accept (e.g., ignore, disregard, delete) any test results if the transmitted serial number (or other identifier) does not match any of the serial numbers in the database and/or the test server otherwise fails to identify/validate the test device. In some cases, when the results are not accepted, the test server may transmit an error message to a user who submitted the test results to the test server by scanning the barcode provided by a diagnostic testing device. The test server may also maintain information indicating the geographic locations (e.g., address, city, state, region, country, etc.) where diagnostic testing devices have been deployed. Associating reported test results with their corresponding diagnostic testing device, therefore, may also allow entities to identify geographic areas or specific locations having relatively more or less rates of infection based on the test results reported by the diagnostic testing devices deployed in those areas or at those locations. Associating reported test results with their corresponding diagnostic testing device may also allow operators to detect potentially malfunctioning diagnostic testing devices by identifying aberrations in the testing reports provided by one testing device relative to other diagnostic testing devices deployed within the same geographic area or at the same location.

Further the test server may be configured to authorize various entities to access some (or all) of the test results based on data access permissions for these entities. Various entities may have different authentication mechanisms including secure password, tokens, codes, and the like for the authentication with a test server that is configured to receive the test results. For instance, a user and a medical professional may be authenticated with the test server and may be authorized to view test-related information as well as personal user-related information, while a government organization may be authenticated with the test server and authorized to receive only test-related information with the personal user-related information removed. Further, a test service provider may be authenticated with the test server to submit the test results but may not be authorized with the test server to view or change the test results, or to view the personal user-related information.

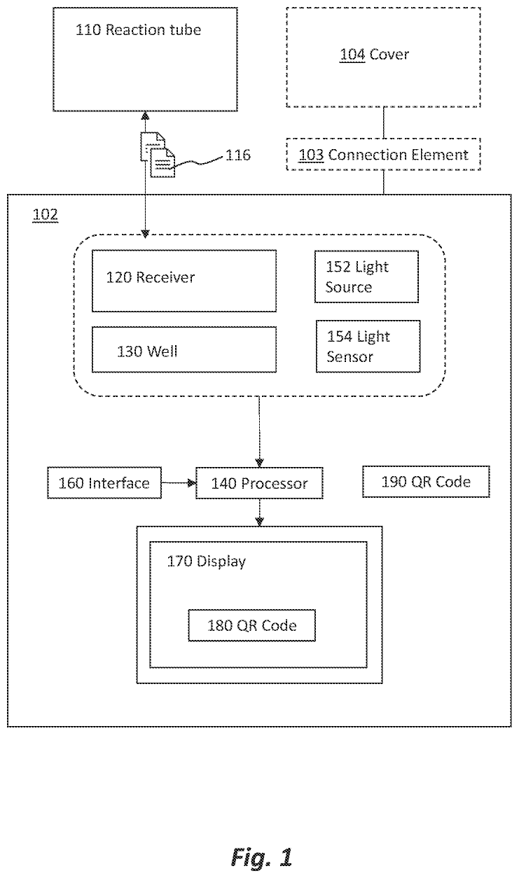

FIG. 1 shows an example of a diagnostic testing device 100 (e.g., a FLOS-LAMP device) operable to amplify an analyte and measure a signal associated with a quantity of the analyte, according to an embodiment. Device 100 further is configured to dynamically generate and display a barcode (e.g., a matrix barcode such as a QR code) for visually communicating test results to a patient, a medical professional, or to an agency for analyzing pathogen transmission within a population. In some cases, the dynamically generated barcode may be a matrix barcode such as a QR code. Other types of barcodes, 2D barcodes, or matrix barcodes may be employed and configured using the techniques described herein. Examples of other types of 2D barcodes and matrix bar codes include AR codes, Aztec codes, Data Matrix, DotCode, EZcode, High Capacity Color Barcode, JAB Code, MaxiCode, PDF417, Qode, ShotCode, and SPARQCode The dynamically generated barcode may be presented via an LCD or other display suitable for presenting the dynamically generated barcode. Device 100 includes a housing 102, which includes a well 130, a receiver 120, a light source 152, and a light sensor 154. Further, housing 102 includes a processor 140, an interface 160, and a display 170 configured to display various test-related information, such as, for example, a dynamically generated barcode 180 as described herein. Additionally, housing 102 includes an optional static barcode 190 (e.g., printed, engraved, or otherwise unchanging barcode) which may be attached to a side of a housing 102 and is uniquely associated with device 100 (e.g., encoding serial number(s), fixed parameters of the device, etc.). The static barcode 190 also may be a 2D barcode or matrix barcode such as a QR code. The static barcode 190 may be scanned to verify possession of a diagnostic testing device and/or that a diagnostic testing device is authentic (e.g., not a knockoff or copycat device). As such, the static barcode 190 may be scanned to initiate an authentication procedure to confirm that an entity (e.g., a particular user, testing facility, and/or the like) is authorized to possess the device. The static barcode 190 also may be scanned to determine and log the geographic location of the diagnostic testing device. In some cases, the information encoded in static barcode 190 (e.g., information identifying device 100) also may be encoded in the dynamically generated barcode 180. Housing 102 of device 100 is configured to receive a reaction tube 110 containing a sample with analyte. The test related information may include a type of test that is performed. In various embodiments, the user name and/or user identification number may be verified by the test service provider prior to performing the test and/or while collecting a sample from the user.

In various embodiments, well 130 may be located within a compartment of housing 102 that may be closed via an optional cover 104 (herein also referred to as a lid 104). Cover 104 may be connected to housing 102 via a connecting element 103, which may be a hinge, a living hinge, or any other suitable connecting element configured to connect cover 104 to housing 102, such that cover 104 can close or open the compartment of housing 102.

In various embodiments, well 130 is configured to receive a reaction tube 110 containing a sample with an analyte. Well 130 may be any suitable opening for receiving at least a portion of reaction tube 110. In an example embodiment, well 130 may have a shape that substantially similar to the shape of at least a portion of reaction tube 110. In an example embodiment, well 130 includes at least a tube holding member and an opening in which reaction tube 110 may be inserted. In an example embodiment, well 130 includes an enclosure having walls, with the walls of enclosure configured to be adjacent (at least partially) to walls of reaction tube 110.

In an example embodiment, reaction tube 110 forms an enclosure containing a sample with an analyte, which, for example, may include a liquid. In an example embodiment well 130 may include one or more windows configured to transmit light from a light emitting source 152. The light source 152 is configured to emit light that when transmitted to the analyte, excites an analyte-emitted light that can be detected by light sensor 154. The information about the detected analyte-emitted light may be analyzed by a processor 140 to determine whether the analyte contains a pathogen (e.g., if the test result is “positive”) or does not contain the pathogen (e.g., if the test result is “negative”).

Processor 140 is configured to be operably coupled to a receiver 120, light emitting source 152 and light sensor 154. In various embodiments, processor 140 is configured to exchange data with receiver 120, source 152 and light sensor 154. For instance, receiver 120 may send data 116 to processor 140, and data 116 may be used by processor 140 for determining operational parameters for source 152 and light sensor 154. In various embodiments, processor 140 may adjust various parameters of the emitted light (e.g., light wavelength, intensity of light, duration of time during which a pulse of light is emitted, a number of pulses of light emitted, or any other characteristics associated with the emitted light). Additionally, processor 140 is configured to activate light sensor 154, and receive data from light sensor 154.

In various embodiments, processor 140 can be, for example, a general-purpose processor, a Field Programmable Gate Array (FPGA), an Application Specific Integrated Circuit (ASIC), a Digital Signal Processor (DSP), and/or the like. Processor 140 can be configured to retrieve data from and/or write data to memory, which can be, for example, random access memory (RAM), memory buffers, hard drives, databases, erasable programmable read only memory (EPROMs), electrically erasable programmable read only memory (EEPROMs), read only memory (ROM), flash memory, hard disks, floppy disks, cloud storage, and/or so forth.

The processor 140 and the associated memory can be communicatively coupled to the light source 152 and/or detector 154 and configured to control a run a diagnostic test by activating various components of device 100. Processor 140 and the associated memory can be operable to receive, process, and/or record signals associated with concentrations of analytes and/or controls. Processor 140 and/or the associated memory can be configured to determine whether the sample contained within the reaction tube 110 is “positive” or “negative” for one or more analytes, according to various methods described in further detail in a provisional patent application 63/275,758, titled “SYSTEMS AND METHODS FOR DETECTING THE PRESENCE OF AN ANALYTE IN A SAMPLE,” filed on Nov. 4, 2021, and a patent application Ser. No. 17/666,338, titled “SYSTEMS AND METHODS FOR DETECTING THE PRESENCE OF AN ANALYTE, SUCH AS SARS-COV-2, IN A SAMPLE,” filed on Feb. 7, 2022, both of which herein incorporated by reference in their entireties. Also, provisional patent application 63/275,758 describes further details of device 100.

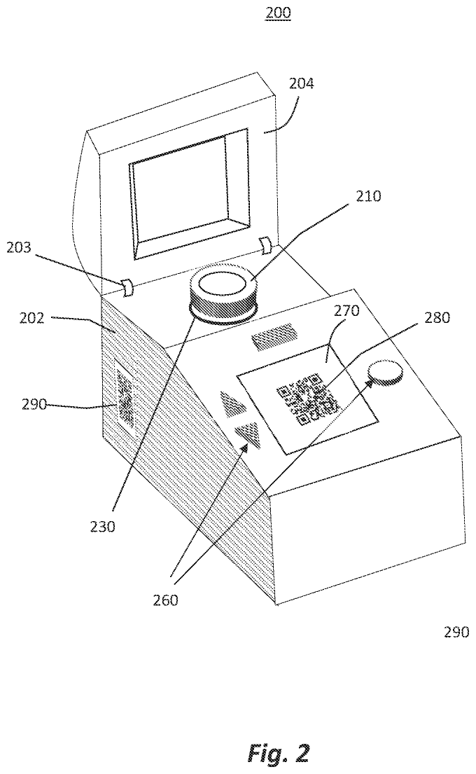

While FIG. 1 shows a block diagram of an example diagnostic testing device 100, FIG. 2 shows an example of a diagnostic testing device 200 which may be an illustrative implementation of device 100. Device 200 includes a housing 202, a cover 204 configured to cover a tube 210 during the assay test. The cover 204 may be attached to the housing 202 via hinge elements 203 (or any other suitable elements). Further, device 200 includes a well 230, display 270, interface elements 260 a dynamically generated barcode 280 displayed on display 270 and a static barcode 290, typically affixed to or engraved on a side of housing 202. In the example shown in FIG. 2, the dynamically generated barcode 280 is a matrix barcode, in particular a QR code. Similarly, in the example shown in FIG. 2, the static barcode 290 also is a matrix barcode, in particular a QR code. In various embodiments, cover 204, hinge element 203, well 230, display 270, interface elements 260, and barcodes 280 and 290 of device 200 correspond to respective cover 104, connecting element 103, well 130, display 170, interface 160, and barcodes 180 and 190 of respective device 100. Further, a reaction tube 210, as depicted in FIG. 2 is shown inserted into well 230, and corresponds to the reaction tube 110, as shown in FIG. 1. As described herein, the form factor of the device 200 and its display 170 may constrain the size of the dynamically generated barcode 280 which, in turn, constrains the size of the payload encoded by the barcode 280

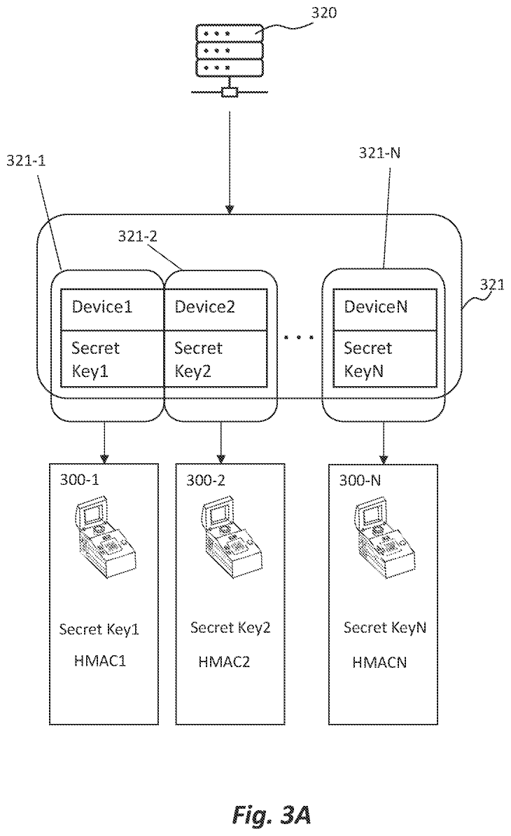

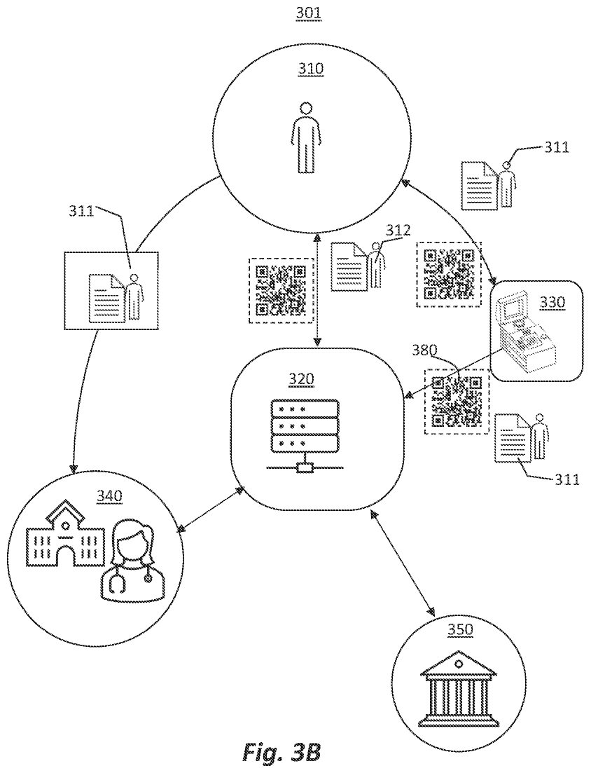

In various embodiments, as described above, device 100 is configured to have an associated static barcode 190. The static barcode 190 may contain information related to device 100. For instance, static barcode 190 may include machine identification information, such as a serial number of the device. In one implementation, the serial number may be encoded using 24 bits. The serial number associated with device 100 may include a combination of a few decimal numbers (e.g., three, four, five, six, seven, eight, nine, ten, eleven, twelve, thirteen, fourteen decimal numbers, and the like), which are allocated for the device during manufacturing, and which uniquely identify the device. Further, static barcode 190 may indicate additional information associated with device 100, such as a type of device 100, a version of device 100, a reporting payload format associated with device 100 (e.g., an indication of the data content and/or structure of data output by dynamic QR code 280), and the like. In some cases, static barcode 190 includes authentication information associated with device 100. For example, such authentication information may include an authentication message that can be used for forming a hash-based (or keyed-hash) machine authentication code (HMAC) to authenticate data associated with test results. The HMAC may be formed based on a secret hidden key associated with device 100, as further described below in relation to FIG. 3A. The HMAC may be used to confirm that data associated with test results is obtained by device 100. The HMAC value protects data integrity generated by device 100, as well as its authenticity, by allowing verifiers to authenticate the device. Generally, the firmware of device 100 may include a secret key known only to the entity authentication received test reports (e.g., the entity that operates the test server). In some embodiments, to obtain the key used for generating the HMAC, the bytes of the secret key stored in the device firmware of the diagnostic testing device may be concatenated with the bytes of the device unique identifier (e.g., the device serial number). In some embodiments, the bytes of the secret key may come before the bytes of the device unique identifier when concatenated. In some embodiments the bytes of the unique identifier may come before the bytes of the secret key when concatenated. A cryptographic hash of the concatenated bytes may then be obtained, e.g., using a cryptographic hash function. Any suitable cryptographic hash algorithm may be employed, e.g., MD5, a Secure Hash Algorithm (SHA) such as SHA-256, SHA-512, or any other cryptographic hash function. The result of the cryptographic hash algorithm may be used as the key for the HMAC generation algorithm. An HMAC may be generated for a payload (e.g., message) comprising the information associated with the diagnostic testing device, e.g., its unique identifier (e.g., serial number), type, version, reporting format, and the like. Any industry-standard algorithms suitable for hashing the payload may be employed. Generating the HMAC may result in a standard 32-byte HMAC. The HMAC may be truncated before being encoded for storage in the static barcode. The payload, together with the truncated HMAC may form the actual content encoded in the QR code. On the server side (e.g., at the test server), the process may be repeated to authenticate and verify the diagnostic testing device. The diagnostic testing device may be authenticated and verified on the server side if the truncated HMAC computed for the received payload-sans-truncated-HMAC (i.e., up to but not including the truncated HMAC) matches the transmitted truncated HMAC. As described herein, a similar process may be used to generate a truncated HMAC for the payload of the dynamically generated barcode 180.

FIG. 3A shows an example diagram for authenticating various diagnostic testing devices 300-1 to 300-N via a test server 320. For example, example test server 320 includes a processor for processing data received by test server 320 (e.g., the processor may be configured to decode data, and compare received data with data stored in a database associated with the processor). In an example embodiment, test server 320 may be associated with a database 321, which includes data that may be stored as column entries 321-1 to 321-N. Each column entry, in this example, includes an associated authentication Secret Key1-KeyN. In various embodiments, Secret Key1-KeyN are used to form associated HMAC as described herein. In an example embodiment, the HMAC may be formed based on a serial number of a device (e.g., device 300-1) and Secret Key1 associated with device 300-1. As shown in FIG. 3A, devices 300-1 to 300-N include respective secret keys Secret Key1-Secret KeyN and device related serial numbers. Secret Key1-Secret KeyN and respective serial numbers can be used by these devices to form HMAC1-HMACN which can be used for authentication purposes with test server 320. Rather than store separate, individual secret keys for each diagnostic testing device, a global secret key may be employed as described herein. The global secret key may be stored in the firmware of each diagnostic testing device and known to the test server (e.g., test server 320) configured to authenticate the diagnostic testing devices. The diagnostic testing devices and the test server may use the global secret key to generate respective HMACs for the individual diagnostic testing devices 300-1 to 300-N as described herein, e.g., using the serial number of the diagnostic testing devices. In this way, even though a global secret is employed for all devices, an HMAC may be bound to a particular diagnostic testing device through the use of the device's serial number that is used to generate the key for the HMAC algorithm.

Reporting test results generated by a diagnostic testing device (e.g., device 300-1) to test server 320 can be performed using the following process. First, device 300-1 performs the diagnostic test and obtain the test results. The diagnostic testing device generates a payload as described herein containing, among other things (e.g., device serial number), the test results. The diagnostic testing device calculates an HMAC for the payload as described herein (e.g., HMAC1) based on a secret key (e.g., Secret Key1) and the device serial number. The diagnostic testing device truncates the HMAC as needed based on the available storage space of the barcode (e.g., a matrix barcode such as a QR code). The diagnostic testing device encodes the payload and the truncated HMAC using a suitable encoding scheme as described herein in order to fit the encoded payload and encoded truncated MAC in the available storage space of the barcode. The diagnostic testing device generates a uniform resource locator (URL) using the encoded payload and the encoded truncated HMAC. The address of the URL is the address of the test server. The diagnostic testing device appends the encoded payload and the encoded truncated HMAC to the address of the test server in the URL. The diagnostic testing device displays the dynamically generated barcode on its display screen. A computing device (e.g., a mobile phone) with suitable scanning equipment (e.g., a camera) scans the displayed barcode. The computing device may be configured (e.g., programmed) to recognize that a barcode was scanned and, based on the scanning, decode the information in the barcode. The computing device also may be configured (e.g., programmed) to automatically navigate to a URL decoded from a scanned barcode. As such, when the computing device decodes the scanned barcode and detects the URL, the computing device may automatically navigate to the address indicated in the URL by generating an HTTP request using the decoded URL. The HTTP request may include both the address of the test server as well as the encoded information appended to the address in the URL. By appending the encoded information to the URL, the barcode is configured to cause (e.g., trigger) the scanning device to automatically provide (e.g., transmit, send, upload) the encoded payload with the test results and the encoded truncated HMAC to a test server (e.g., test server 320) via the HTTP request that is generated based on (e.g., in response to) the scanning. Based on receiving the HTTP request with the URL, the test server may perform an authentication procedure to authenticate the reported test results. As described herein, the test server may generate a truncated HMAC for the encoded payload in the URL (e.g., using the secret key and the device serial number obtained from the encoded payload). The test server may compare the received truncated HMAC to the generated truncated HMAC. If the two HMACs match, the test server may determine the received report is authentic and valid. If not, the test server may discard the received report and may report an error via a suitable interface (e.g., via a webpage, a smartphone application, and the like). The test server may store the test results for valid reports that it receives. The test server may also evaluate the additional information accompanying the test results sent in the report to assess the operating status of the diagnostic testing device that performed the test in order to determine whether the received test results are reliable. For example, as described herein, a received report may include information indicating the operation status of the diagnostic testing device (e.g., various statistics relating to the tests performed, error codes, and the like). Based on such information, the test server may determine whether the test results are reliable (e.g., true positive, true negative) or unreliable (e.g., false positive, false negative). In some embodiments, the test server may store test results deemed to be reliable and discard (e.g., not store, delete, ignore) test results deemed to be unreliable. In some embodiments, the test sever may store test results deemed to be reliable in one data storage location (e.g., one database) and may store test deemed to be unreliable in another data store (e.g., another database). In some embodiments, the test server may store the test results with a determined measure of reliability. The test server may determine a measure of reliability for received test results based on the accompanying information indicating the operating status of the diagnostic testing device (e.g., a current operating status during the current test and/or one or more historical operating statuses for previously performed tests).

FIG. 3B shows a system 301 which includes a user 310, a test service provider 330, a medical professional 340 and a government or health agency 350 interacting with test server 320. In an example embodiment, user 310 may register with the test server 320 by providing personal user information 312 which may include a user first and last name, address, phone, email, data of birth, race, ethnicity, medical history, place of birth, and the like. In some cases, only login information may be selected by the user (e.g., a user id and/or user email, and/or user phone, and password) to register with the test server 320. Upon registering, test server 320 may provide an account number (or any other suitable user identification) for user 310. Further, user 310 may send at least some of the personal user-related information 311 to the test service provider 330. For example, personal information 311 may include user first and last name, user phone, or user date of birth. In some cases, the personal user-related information 311 may be an account number (or any other user identification provided by test server 320) associated with the user 310. Communicating user-related information 311 to the test service provider 330 allows for the test service provider 330 to link test results data with user 310 (or user identifier), thus, allowing the test server 320 to associate test results with the user (or user identifier) and facilitating access of the test results by user 310.

In cases when user 310 performs functions of test service provider 330, or when user 310 receives a barcode (e.g., a matrix code such as a QR code) from test service provider 330 (e.g., dynamically generated barcode 180, as described above) formed after performing a diagnostic test, user 310 may scan the barcode. The barcode may encode a link that can be automatically executed by a smartphone operating system (e.g., Android or iOS) as described herein to direct a web browser to a website 320 hosted by or associated with the test server 320. The website may provide a web portal associated with user 310 (e.g., test service provider 330) that includes form fields, graphical user elements, and the like, facilitating user 310 to provide user-related information 311 (e.g., test services provider information, patient information, and the like).

Further, user 310 (e.g., an individual being tested for infection) provides a test sample (e.g., a nose swab, a throat swab, a blood sample, and the like) to test service provider 330. Test service provider 330 can introduce the test sample into a reaction tube (e.g., in a reaction tube 110, as shown in FIG. 1). The reaction tube 110 can contain reagents capable of amplifying an analyte if present in the test sample and/or dyes or other suitable markers to aid in the detection of the analyte.

Test service provider 330 may perform the test using a diagnostic device that is configured to display dynamically generated barcode 380 (e.g., a matrix code such as a QR code) on a display screen (e.g., a barcode similar to or the same as dynamically generated barcode 180, as shown in FIG. 1). After completion of the test, dynamically generated barcode 380 is scanned by the test service provider 330 (or user 310, if user 310 receives dynamically generated barcode 380 from test service provider 330), for example, using a smartphone or other suitable device. As described herein, barcode encodes a URL. The test service provider 330 (or user 310) can scan dynamically generated barcode 380 to obtain the URL, and the smartphone operating system may automatically direct a browser application to the URL obtained from dynamically generated barcode 380. As described herein, the address of the URL can be associated with a webpage that provides a web portal (e.g., a reporting portal) associated with the test service provider 330. FIG. 3B shows that dynamically generated barcode 380 may optionally (as indicated by dashed border around dynamically generated barcode 380) be displayed to user 310, and then data associated with the ally generated barcode transmitted to test server 320 by scanning dynamically generated barcode 380 by user 310. When data associated with dynamically generated barcode 380 is transmitted to test server 320 from user 310, the data associated with dynamically generated barcode 380 may not be communicated to test server 320 from test service provider 330. In some cases, when data associated with dynamically generated barcode 380 is communicated to test server 320 by test service provider 330, the data associated with dynamically generated barcode 380 may not be communicated to test server 320 by user 310.

The dynamically generated barcode may encode a URL that includes parameters (also referred to as arguments) as described herein that encode test data related to the test performed by the diagnostic testing device. In this way as described herein, when a browser is directed to the URL, a server hosting the webpage may receive the parameters, decode the test data, and store the information in one or more databases associated with test server 320, thus, creating a test result data record. Further, a timestamp indicating a time when the test result data record has been created may be stored with the associated test result data record.

FIG. 3B further shows that user 310 may transmit user-related information 311 to a medical professional 340, and medical professional 340 may access test results by authenticating with test server 320. In an example embodiment, medical professional 340 may be registered with test server 320 and may obtain the test results of user 310 based on user-related information 311 received from user 310.

Additionally, as shown in FIG. 3B, a government agency 350 (or similar organization, such as health organization, world health organization, charity, non-profit organization, private organization, a group of individuals, another individual, and the like) may be configured to inquire about test results from test server 320. Government agency 350 may also be registered with test server 320 and may have authorization to receive at least some information about test results of one or more users (e.g., if such authorization is granted or mandated by law). In one example, government agency 350 may not have access to personal user information but may receive information indicating individual positive/negative results and/or overall test positivity rate within a population. For example, government agency 350 may receive information of positive test results (or negative test results) at a given location (or within a given region) during a given time interval. For instance, the government agency 350 may receive information of positive COVID-19 cases and negative COVID-19 cases in a specified city (e.g., New York city) for a given day, given hour, or given minute. In various embodiments, when a dynamically generated barcode (e.g., dynamically generated barcode 380) is scanned, the test results associated with the dynamically generated barcode may be sent from the test server to the government agency (e.g., if such reporting is mandated by law). In this way, obtaining test results by a user and reporting test results to a governmental agency can be linked, which can reduce or eliminate reporting biases. It should be noted that the test results associated with dynamically generated barcode 380 may not be processed more than once if the same dynamically generated barcode 380 is scanned more than once. For example, if dynamically generated barcode 380 has been previously scanned, and the test results associated with dynamically generated barcode 380 were successfully processed, the data obtained by scanning dynamically generated barcode 380 again may not be processed by test server 320 (e.g., test server 320 may not send the data to the government agency when dynamically generated barcode 380 scanned the second time, the third time, and the like). However, if scanning dynamically generated barcode 380 resulted in a failure of processing the test results by test server 320 (an indication of the failure may be communicated to a user who is scanning dynamically generated barcode 380 via a suitable user interface associated with the scanning application employed by the user), test server 320 may be configured to accept new test results obtained by subsequent scanning of dynamically generated barcode 380.

Returning to FIG. 1, device 100 includes an interface 160 configured to interface with processor 140. Interface may include one or more buttons, a touchscreen, a touchpad, a joystick, and the like. For example, a particular implementation of interface 160 is shown in FIG. 2 by interface elements 260, which may include several buttons. In one embodiment, interface 160 may allow a user to input various parameters associated with a diagnostic test (e.g., interface 160 may allow the user to input a type of the diagnostic test to be performed, or other parameters associated with the diagnostic test, such as, for example, a wavelength of light that may be emitted by light source 152 to interrogate the sample). Further, interface 160 may allow user to open/close a cover of the device 100, or to allow users to interact with device 100 in any other ways. For instance, interface 160 may be used to select the information for display via a display screen 170.

In various embodiments, display screen 170 is configured to display any suitable information for a test service provider. For example, prior to performing a diagnostic test, the display screen 170 may display information related to a type of the diagnostic test to be performed. For example, such information may be displayed in order to receive a confirmation from the test service provider and/or the user that the correct diagnostic test is selected.

In various embodiments, as discussed above, display screen 170 may further present results of the diagnostic test encoded in a dynamically generated barcode 180 (e.g., a matrix code such as a QR code) that may not be easily readable by a person. In various embodiments, dynamically generated barcode 180 may be scanned using any suitable electronic device e.g., an electronic device containing a camera, such as a smartphone. In some cases, other electronic devices containing a camera, or a scanner may be used (e.g., laptops, desktops, scanners, and the like). Note that in some instances, default camera and/or scanning apps can be operable to transmit the encoded data to test server 320 (e.g., by launching a default web browser, populating the address bar with an encoded URL with arguments, and directing the web browser to the URL) such that no special-purpose software may be necessary, and dynamically generated barcode 180 may be scanned in the same way as scanning of any other barcode (e.g., dynamically generated barcode 180 may be scanned using a code scanning application and/or feature of a smartphone).

Dynamically generated barcode 180 may be any suitable barcode for encoding test related information including, for example, matrix codes such as a QR code and other types of 2D barcodes. For example, dynamically generated barcode 180 may be a model 1 or model 2 QR code. For instance, dynamically generated barcode 180 may be a version 1 through version 40 QR code. For instance, version 1 QR code has the data matrix of size 21×21 elements, version 2 QR code has the data matrix of size of 25×25 elements, version 3 QR code has data matrix of size of 29×29 elements, version 4 QR code has data matrix of size of 33×33 elements, and the like. In some cases, QR code of version 4 may be sufficient to represent all the necessary data related to a test result. Alternatively, in some cases, a version 40 of QR code may be used (version 40 QR code has data matrix of size 177×177 elements). Dynamic QR code 180 may be of any suitable level L (low), M (medium), Q (quartile), or H (high). For example, level L allows to restore 7% of the data when dynamic QR code 180 is damaged (or poorly scanned or captured), and level H allows to restore up to 30% of the data when dynamic QR code 180 is damaged (or poorly scanned or captured). In various embodiments, a highest level of error correction available (level H) may be used to allow for errors when capturing dynamic QR code 180 via an auxiliary device (e.g., via a camera of a smartphone). The highest error correction (level H) may be selected to afford the greatest robustness. As described herein, the display screen size of the diagnostic testing device and/or the resolution used for each barcode unit/module may limit the size of the possible barcodes that may be presented on the display screen. For example, a display screen size of 150×150 pixels and a resolution of 4×4 pixels per barcode unit/module may constrain the size of any QR code to being no larger than 37×37 elements (i.e., version 4 QR code at 33×33) which would result in the displayed QR code having a size of 132×132 pixels (150 pixels divided by 4 pixels per barcode unit/module equals 132 pixels). As also described herein, the size of the barcode (e.g., the size of a QR code) used along with a selected level of error correction may limit the amount of information (e.g., the payload of the barcode) that can be included in the barcode. It will be appreciated that different display screen sizes, pixel resolutions, barcode sizes, and the like may be employed depending on various factors including, for example, form factor (e.g., footprint) of the diagnostic testing device, costs, aesthetics, and other desired or required design considerations that impact the visual presentation capabilities of a given diagnostic testing device. As described herein, various techniques may be employed to maximize the amount of information that can be stored in a dynamically generated barcode within the constraints of a given implementation.

The selected encoding scheme may be one example of a technique to help maximize the amount of information included in a dynamically generated barcode (e.g., a matrix code such as a QR code). In various embodiments, a dynamically generated barcode (e.g., a dynamically generated QR code 180) may include numerical-only encoding, alphanumeric encoding, binary encoding, or kanji/kana encodings, as known in the art. In one example implementation, dynamically generated barcode 180 may include alphanumeric encoding. As described herein, the alphanumeric encoding may provide a tradeoff between information density and sufficient number of characters to express a URL that can be used to provide the test information in a visual manner with sufficient markers of authenticity and reliability. As described herein, the pixel resolution (e.g., 4×4 pixels per barcode unit/module/“dot”) may serve as a tradeoff between the size of the display screen of the diagnostic testing device and reserving enough space on the display screen for additional visual elements such as borders and supporting instructional text. In some embodiments, the display screen may present only the dynamically generated barcode in order to maximize the size of the barcode presented. In some embodiments, the display screen may simultaneously display the dynamically generated barcode with one or more additional elements (e.g., ornamentation such as borders, instructions, control options, and the like) in which case the maximum size of the dynamically generated barcode is constrained by both the size of the display screen as well as any additional elements simultaneously presented. As described herein, the barcode configuration and display screen may include various other configurations and sizes and adjustments as known and used in the art.

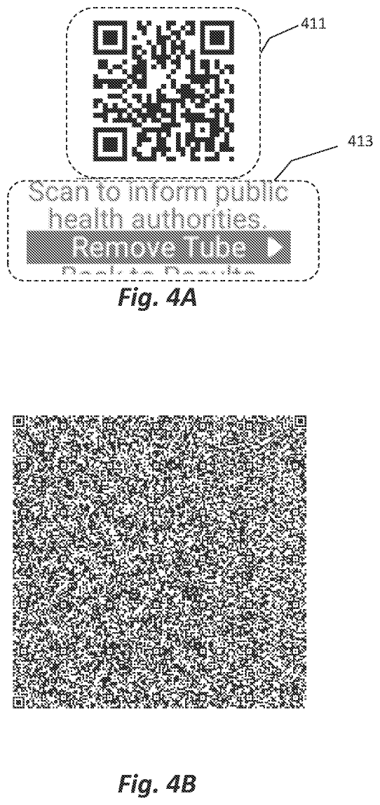

FIGS. 4A and 4B show possible examples of dynamically generated barcodes (e.g., barcode 190 or barcode 180). The example dynamically generated barcodes shown in FIGS. 4A and 4B are matrix barcodes, in particular QR codes. For example, FIG. 4A shows an example of display screen 170 which includes a QR code 411, and an additional text 413 adjacent to the QR code 411. As shown, QR code 411 is a version 4 QR code. In some cases, as shown by FIG. 4B, QR code 415 may be a version 40 QR code and include more information that version 4 QR code.

The URL encoded by a dynamically generated barcode (e.g., a matrix code such as a QR code) may be generated in a manner that maximizes the size of the payload appended to the address (e.g., the hostname) of the URL. In various embodiments, the URL includes a prefix (HTTPS://) followed by a website address domain. To maximize the size of the payload in the URL, the web address (e.g., the domain name and/or the fully qualified domain name). In an example embodiment, a minimum number of characters may be employed for the website domain and/or the top-level domain (e.g., three characters, four characters, five characters, six characters, seven characters, eight characters, nine characters, ten characters, and the like). For example, a top-level domain (i.e., domain suffixes) that includes two characters may be employed. Such domains may correspond to country code domains (e.g., “.to”, “.tw”, “.uk”, “.us,” and the like). Similarly, a two-letter domain name may be employed, thereby resulting in only a five-character fully qualified domain name (e.g., xx.xx) and resulting in only a thirteen-character URL (e.g., https://xx.xx) that a payload with encoded test results and accompanying information may be appended to. A single forward slash (“/”) may be employed as a delimiter between the address and the payload in the URL. Other delimiters may be employed.

In some embodiments, the payload appended to the address in the URL may include information used to help decode the payload received via the URL. Such information may be indicated using one or more characters. For example, a single selected character (e.g., “A” or “B” or “C” or “D”) may indicate one or more of: a role of the barcode that was scanned (e.g., if the barcode is a dynamically generated barcode used to provide the test results or if the barcode is a static barcode used to provide information about a diagnostic testing device), an encoding character set used to encode the test results, accompanying data, HMAC, and the like as described herein, an indication of a version of the barcode scanned (e.g., which QR code version), or any other information that is related to the barcode that was scanned (e.g., a level of barcode, and the like). Any suitable encoding character set may be employed to encode the test results, accompanying data, HMAC, and the like. The encoding character set may be an alphanumerical set corresponding to a particular base-n encoding. For example, for base-10 encoding, numerical characters (0-9) are used; for base-16, hexadecimal characters are used such as characters (0-9) as well as (a-f) characters respectively corresponding to numbers (10-15). Further, base-32 encoding can be used (which uses all the letters of the alphabet and numbers 2-7) or any other suitable base encoding (e.g., base-41 encoding, base-43 encoding, base-48 encoding, base-64 encoding, and the like). In various embodiments, for base-43 encoding, characters (0-9, A-Z, $, *, +, −, ., /, :) may be used, and for base-41 encoding, characters (0-9, A-Z, $, *, +, −, .) may be used. The encoding scheme used to encode the test results, accompanying data, HMAC, and the like, may be selected in order to minimize the size of the payload appended to the address in the URL. For example, a base-43 or base-41 encoding may be used to minimize the size of a payload where a matrix barcode (e.g., version 4 QR code) is used to visually present the test results with sufficient markers of authenticity and reliability.

To obtain the encoded payload for the URL, the selected encoding scheme is used to encode a bit sequence corresponding to test results report. The bit sequence may be referred to as a bit-packed data structure (BPDS) and may be the result of a concatenation of various bit sequences respectively corresponding to the report, e.g., the test results themselves, the accompanying information about the diagnostic testing device, the HMAC, and the like. This concatenated bit sequence, BPDS, may represent a very large integer that is then encoded using the selected encoding scheme which reduces the size of the data from relatively long sequence of ones and zeros to a relatively short sequence of characters from the selected encoding scheme. The BPDS may include a “core” that includes the test results, information about the test run (e.g., run number, type), information about the diagnostic testing device (e.g., operating status, error code), and other information as described herein.

For example, a BPDS core may include (1) the format or version of the reporting payload (which is encoded using only a few bits, e.g., 1, 2, 3, 4, 5, 6, 7, 8, 9, or 10 bits). In order to minimize the size of the BPDS core, only one or two bits may be used to indicate the format or version of the reporting payload. The format or version of the reporting payload format may be associated with the selected encoding scheme employed. In some embodiments, in order to minimize the size of the BPDS core, a minimum number of bits may be used to indicate the format or version of the reporting payload; in other embodiments, more bits may be used

A BPDS core also may include (2) an indication of a type of diagnostic testing device (e.g., a product identifier) used to perform the diagnostic test (which also may be encoded using only a few bits, e.g., 1, 2, 3, 4, 5, 6, 7, 8, 9, or 10 bits). In order to minimize the size of the BPDS core, a minimum number of bits may be used to indicate the type of diagnostic testing device.

A BPDS core may also include (3) a serial number of the diagnostic testing device that performed the diagnostic test (which may be encoded using a few bits to a few dozen bits, e.g., 10-48 bits). In some embodiments, in order to minimize the size of the BPDS core, a minimum number of bits may be used to indicate the serial number of the diagnostic testing device; in other embodiments, more bits may be used.

A BPDS core may include (4) a test run number which identifies a running count of the number of diagnostic tests performed by the diagnostic testing device. The test run number may be persistently stored in a memory of a diagnostic testing device (e.g., diagnostic testing device 100) and may monotonically increase each time a new sample is inserted, and the new analysis is made (i.e., whenever a diagnostic test is performed). The test run number may be encoded using a few to a few dozen bits (e.g., 10-36 bits). In some embodiments, in order to minimize the size of the BPDS core, a minimum number of bits may be used to indicate the test run number; in other embodiments, more bits may be used.

A BPDS core may include (5) an indication of a type of test that the diagnostic testing device performed. For example, the type of the test may be a COVID test, a Flu test, a combination of COVID and a Flu test, or any other suitable test that can be performed by the diagnostic testing device (e.g., diagnostic testing device 100) (e.g., an RSV test or a Streptococcus test). The indication of the type of test may be encoded using a few bits (e.g., by 1, 2, 3, 4, 5, 6, 7, 8, 9, or 10 bits). In some embodiments, in order to minimize the size of the BPDS core, a minimum number of bits may be used to indicate the indication of the type of test; in other embodiments, more bits may be used.

In some cases, even the same type of test (e.g., COVID test) may have different sub-types (e.g., versions), and a diagnostic testing device (e.g., diagnostic testing device 100) may be configured to adjust the test parameters depending on a particular version of the particular type (e.g., on a particular version of a COVID test). For instance, for a first type of COVID test a first wavelength, intensity, or a duration of illumination of light from the light source 152 may be selected, and for the second type of COVID test a second wavelength, intensity, or a duration of illumination of the light may be selected. In some cases, any other suitable parameters may be adjusted for different versions of the same type of the test. For instance, when device 100 includes a heating element for heating the analyte, the heating temperature of the heating element may vary depending on the version of the test performed. In some cases, the version of the test that is being run may differentiate from other versions of the test based on a type of kit that is being used for the test (e.g., the amount of analyte, the type of analyte, the type of reaction tube 110, the transparency of walls of reaction tube 110, and the like). In various embodiments, the data about a type of test that is being run may include information about a sub-type of the test for the particular type of the test that is being run. The type of test and the test version may be encoded by a few bits (e.g., by 1, 2, 3, 4, 5, 6, 7, 8, 9, or 10 bits). In some embodiments, in order to minimize the size of the BPDS core, a minimum number of bits may be used to indicate the test type and test version (sub-type); in other embodiments, more bits may be used.

A BPDS core may also include (6an indication of the outcome of the test. The outcome may include a “positive,” a “negative,” an “inconclusive,” a “malfunction,” result, and the like. In some cases, the result may be characterized by a probability of being positive or negative. In some cases, the results may be a numerical value (e.g., a glucose level in a blood sample, a cholesterol level, and the like). The outcome of the test may be encoded by a few bits (e.g., by 1, 2, 3, 4, 5, 6, 7, 8, 9, or 10 bits). In some embodiments, in order to minimize the size of the BPDS core, a minimum number of bits may be used to indicate the outcome of the test; in other embodiments, more bits may be used.

A BPDS may also include one or more statistics indicating a current and/or historical operational state of the diagnostic testing device. For example, the BPDS may include an indication of historical outcomes of diagnostic tests performed by a diagnostic testing device (e.g., diagnostic testing device 100). For instance, a BPDS may indicate a quantity of positive results, a quantity of negative results, a quantity of inconclusive results, a quantity of canceled tests, and/or a quantity of malfunctions based on previously performed diagnostic tests (e.g., based on previously performed tests during an interval of time, such as during the previous week). In an example implementation, a diagnostic testing device may use separate individual bit sequences to indicate the respective counts of positive results, negative results, inconclusive results, cancelled tests, and/or malfunctions. Such bit sequences may include a few bits or a few tens of bits (e.g., 5-20 bits). In some cases, 9 or 10 bits may be sufficient to provide an indication of such testing statistics associated with a diagnostic testing device. In some embodiments, in order to minimize the size of the BPDS core, a minimum number of bits may be used to indicate individual testing statistics; in other embodiments, more bits may be used (e.g., to avoid bit overruns). Bit overruns may be possible depending on the size of the bit sequence used to store a given testing statistic (e.g., if 9 bits are employed to store the number of negative test results, the bit sequence would rollover after 512 negative results and upon the 513^{th} negative result). Bit overruns, however, may be detected by comparing the test run number to the reported testing statistics.

In some cases, when multiple tests are performed simultaneously (e.g., when both COVID and flu tests are performed simultaneously or consequently), the outcomes of the test may be represented as a pair of outcomes. For instance, for a test of COVID and flu (herein, such test is referred to as a binary COVID-flu test with the COVID and the flu test referred to as a subtests), the outcome may be {COVID, flu}={positive, positive}, {COVID, flu}={positive, negative}, and the like. Besides a binary test, a ternary test can be performed, i.e., the test having three subtests, a quaternary test, i.e., the test having four subtests, or a test having more than four subtests. Thus, at least for some tests, more information may be encoded in the QR code. In one example implementation, a data record may be used to indicate that both outcomes occur simultaneously. Further, another data record may be used to indicate that a binary COVID-flu test (or any other binary test, has been carried out, and for each subtest, dynamic QR code 180 may allocate a data record indicating outcome of that subtest and statistics associated with the outcome for that subtest.

In some cases, dynamic QR code 180 may encode data indicating statistics for subtests when more than one subtest was performed. For example, when binary COVID-flu test is performed for a given sample, the QR code may encode the number of positive outcomes for COVID when the binary COVID-flu test is performed as a first statistic (s1). Further, the number of positive outcomes for COVID when only a COVID test is performed may be encoded in the QR code as a second statistic (s2), that is different from the first statistic (s1). Further, if binary COVID-flu test is performed, each data record for an associated subtest may be encoded by sufficient number of data bits to communicate the subtest-related information.

In some embodiments, a bit sequence may be employed to indicate a positive result for each of multiple targets (t₁-tₙ) being tested for. For example, positive test results in a BPDS core may include a bit sequence of n bits indicating for each of the n possible targets detectable for the diagnostic test that was performed. Each bit in the bit sequence may correspond to one of the targets where, e.g., a one (“1”) indicates a positive result for the target and a zero (“0”) indicates a negative result for the target. In some embodiments, the test result may be indicated as negative if none of the n targets are detected, but a control signal such as an internal amplification control (IAC) is detected. In some embodiments, the test result may be indicated as inconclusive if none of the n targets is detected and the IAC is not detected.

A BPDS core may also include an indication of an amount of time (e.g., a number of seconds) that elapsed before detecting the internal amplification control (IAC) signal. In some embodiments, the BPDS core may include the indication of a number of seconds before detecting the IAC signal based on obtaining a positive result or based on obtaining a negative test result. The IAC is a nontarget DNA sequence present in the analyte, which is coamplified simultaneously with the target sequence. In a diagnostic test (e.g., PCR, loop-mediated isothermal amplification, antigen test, and the like) without an IAC, a negative response (no band or signal) could mean that there was no target sequence present in the reaction. However, it could also mean that the reaction was inhibited, (e.g., due to malfunction of thermal cycler, when PCR test is used), incorrect reagent mixture, poor DNA polymerase activity (when using PCR test), or due to the presence of inhibitory substances in the sample matrix. Conversely, in a diagnostic test with an IAC, a control signal is expected even though there is no target sequence present. This can reveal failure of the diagnostic test when control signal is not produced. If no control signal is observed when IAC is present, the elapsed time may be evaluated to zero. The indication of the elapsed time may be encoded using a few data bits (e.g., 10, 12, 14, 16 data bits, and the like). In some embodiments, in order to minimize the size of the BPDS core, a minimum number of bits may be used to indicate the elapsed time; in other embodiments, more bits may be used.

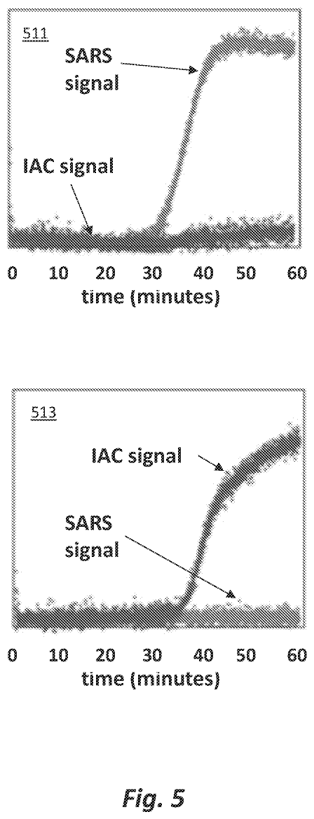

FIGS. 5A and 5B show example SARS signals and IAC signals for a positive SARS-CoV-2 test 511 and for a negative SARS test 513. For the positive SARS test 511, the SARS signal is significantly higher than the IAC signal, and for the negative SARS test 513, the SARS signal may be smaller than IAC signal.

As described herein, a BPDS core may include an indication of an amount of time that elapsed before detecting a particular one of outcomes such as positive, negative, malfunction, inconclusive, or canceled outcomes. Such indication may be also encoded using a few data bits (e.g., 10, 12, 14, 16 data bits, and the like). In some embodiments, in order to minimize the size of the BPDS core, a minimum number of bits may be used to indicate the elapsed time; in other embodiments, more bits may be used.

When the test results indicate “malfunction,” a BPDS may also include a data code indicating the reason for the malfunction. The number of bits used to indicate an error code may be based on the number of possible reasons for malfunction. In some embodiments, in order to minimize the size of the BPDS core, a minimum number of bits may be used to indicate an error code; in other embodiments, more bits may be used. The enumerated reasons may include the parameters summarized in a Table 1 below.

TABLE 1
possible reasons for malfunction

| | |
|---|---|
| FAIL_TO_WARM_UP_IN_TIME | indicates that analyte did not warm up |
| | properly |
| TUBE_OUT_OF_SPEC_DURING_TEST | indicates the absence of a reaction tube |
| | during a test, out of tolerance reaction tube |
| | opacity, or that the reaction tube is |
| | improperly positioned |
| LID_OUT_OF_SPEC_DURING_TEST | indicates that lid (cover) of the device is not |
| | properly closed during the test |
| TUBE_TEMP_OVERHEAT | indicates that reaction tube is overheated |
| | during the test |
| LID_TEMP_OVERHEAT | indicates that lid (cover) of the device is |
| | overheated during the test |
| TUBE_TEMP_I2C_FAILURE | Tube temperature sensor communication |
| | failed |
| LID_TEMP_I2C_FAILURE | Lid temperature sensor communication |
| | failed |
| SPECTRAL_I2C_FAILURE | Optical sensor communication failed |
| BLUE_SENSOR_LOW | Signal on the blue optical channel is too low |
| BLUE_SENSOR_HIGH | Signal on the blue optical channel is too |
| | high |
| YELLOW_SENSOR_LOW | Signal on the yellow optical channel is too |
| | low |
| YELLOW_SENSOR_HIGH | Signal on the yellow optical channel is too |
| | high |
| GREEN_SENSOR_LOW | Signal on the green optical channel is too |
| | low |
| GREEN_SENSOR_HIGH | Signal on the green optical channel is too |
| | high |
| RED_SENSOR_LOW | Signal on the red optical channel is too low |
| RED_SENSOR_HIGH | Signal on the red optical channel is too high |
| BLUE_GREEN_RATIO_LOW | Signal ratio between Blue/Green is too low |
| BLUE_GREEN_RATIO_HIGH | Signal ratio between Blue/Green is too high |
| YELLOW_RED_RATIO_LOW | Signal ratio between Yellow/Red is too low |
| YELLOW_RED_RATIO_HIGH | Signal ratio between Yellow/Red is too |
| | high |
| BLUE_SENSOR_PAIR_MISMATCH | The two blue sensing zones are equivalent |
| YELLOW_SENSOR_PAIR_MISMATCH | The two yellow sensing zones are |
| | equivalent |
| GREEN_SENSOR_PAIR_MISMATCH | The two green sensing zones are equivalent |
| RED_SENSOR_PAIR_MISMATCH | The two red sensing zones are equivalent |
| UNHANDLED_ASSAY_FINISH_CODE | User cancelled the test. The device can not |
| | issue a result. |
| SPECTRAL_SMUX_SETUP_FAIL | Multiplexer set-up failed |
| ASSERT | indicates that an assertion error has |
| | occurred based on an assertion condition |

When an ASSERT failure is encountered, the ASSERT failure may be due to an assertion error in a software executed by a processor of a diagnostic testing device. In such case, ASSERT failure may indicate information that allows to identify the assertion error. For example, the assertion error may be identified if a line number of the software at which assertion error is occurred is identified and a first few characters of the file in which the assertion error has occurred are identified to locate the file with the assertion error. When a ASSERT failure occurs, a BPDS may include an indication of the line number at which the ASSERT occurred and an indication of the file (e.g., the first n characters) in which the assert occurred. In an example embodiment, the line number may be encoded using, for example 16 bits of data, and the first few characters of the file (e.g., a first three, four, five, six, etc. characters) may be encoded using 32 bits of data. In some embodiments, in order to minimize the size of the BPDS core, a minimum number of bits may be used to indicate the line number and the characters of the file; in other embodiments, more bits may be used.

In various embodiments, for inconclusive outcomes, a BPDS core may include no additional information, e.g., in order to minimize the size of the BPDS core. In other embodiments, a BPDS core may include additional information with the indication of an inconclusive outcome.

A BPDS core may include zero-padding to achieve byte alignment.

In some embodiments, a BPDS may include both the BPDS core as described herein as well as an HMAC for the BPDS core obtained using a secret key associated with the diagnostic testing device as described above. In some embodiments a diagnostic testing device may lack (i.e., not include) an internal clock, e.g., to minimize the complexity of the device, the cost of the device, the form factor of the device, and the like. In other embodiments, a diagnostic testing device, optionally, may include a clock. A BPDS core may include one or more timestamps provided by any clock included at a diagnostic testing device.

In various embodiments, the HMAC may be truncated to fit within the available space of the dynamically generated barcode. While a truncated HMAC may be relatively less secure than an HMAC that is not truncated, a truncated HMAC may still provide sufficient cryptographic strength. In some cases, the HMAC may be truncated to fewer than 32 bytes. For example, in some cases, the HMAC may be truncated to 11 bytes. In some cases, a BPDS core may include 21 total bytes which, when concatenated with 11 bytes of the HMAC yields a BPDS having 32 bytes to be encoded for the URL.

In various embodiments, for a static barcode (e.g., static barcode 190 attached to a housing of a device), may include 4 bytes, thereby resulting in BPDS having 15 bytes in total when BPDS includes static barcode information (the BPDS includes HMAC truncated to 11 bytes and 4 bytes of the static barcode). In various embodiments, having HMAC (e.g., truncated HMAC) provides an assurance that the barcode in question is associated with a bona fide device 100.

Additionally, in some implementations, a barcode may further include additional encoded data. For example, the additional encoded data may include an amount of time (e.g., seconds, or minutes) that the barcode has been displayed on the screen, prior to being refreshed or updated. The update of the barcode may happen periodically. Such information combined with the information about the time at which a scan was received by a portal server (e.g., test server 320, as shown in FIGS. 3A and 3B) may provide an indication as to when, in real time, the test was begun. As described herein, in some cases, device 100 may optionally include an internal clock, and a timestamp associated with the internal clock may be also encoded in the QR code.

As also described herein, in some embodiments, a diagnostic testing device may lack (i.e., not include) a wireless communication interface (e.g., a radio, transmitter, receiver, transceiver, antenna, and the like) that is used to report the test results and accompanying information. Excluding networking interfaces (e.g., wired and wireless networking interfaces) means that no network vectors are available to attack a diagnostic testing device In some embodiments, a diagnostic testing device may include a physical (e.g., wired) communication interface (e.g., Ethernet, USB, serial, telephone, and the like) or wireless communication interface (e.g., cellular, Wi-Fi, Bluetooth, and the like) for other purposes, e.g., to receive over-the-air firmware updates, control signals (e.g., factory reset signal, shutdown/disable signal), and the like. A diagnostic testing device may lack (i.e., not include) a wired communication interface (e.g., Ethernet, USB, serial, telephone, and the like) wireless communication interface (e.g., a radio, transmitter, receiver, transceiver, antenna, and the like), e.g., to minimize the complexity of the device, the cost of the device, the form factor of the device, and the like.

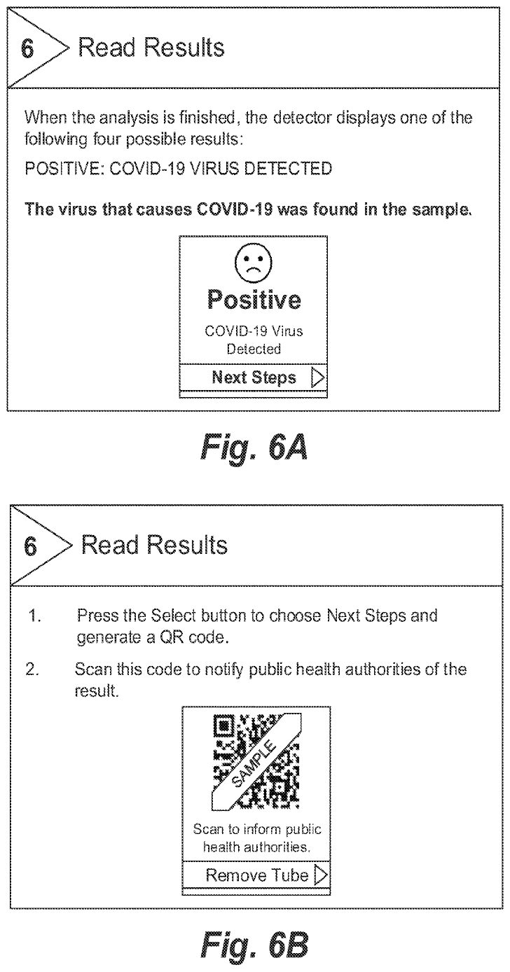

FIGS. 6A and 6B show examples of information that may be displayed on a display 170 of the device 100. For instance, FIG. 6A shows an example display reporting a positive test result. (Note, in some instances, particularly when sample analysis occurs at a remote location such as a lab, a test service provider may not have access to an outcome of the test result; in other instances, such as when the test is performed in the patient's presence, the test service provider may be able to observe the outcomes of test results on the display of the device). FIG. 6B shows an example display for prompting a test service provider to interact with interface 160 (e.g., press a button similar to interface element buttons 260, as shown in FIG. 2) to dynamically generate barcode 180 (e.g., a matrix code such as a QR code). Once dynamically generated barcode 180 is generated, the test service provider and/or a user may use a barcode scanning device (e.g., a smartphone) to scan and transmit data encoded in the dynamically generated barcode 180 to a test server (e.g., test server 320, as shown in FIGS. 3A and 3B) such that the data can be stored in a data store and/or database associated with a user account.

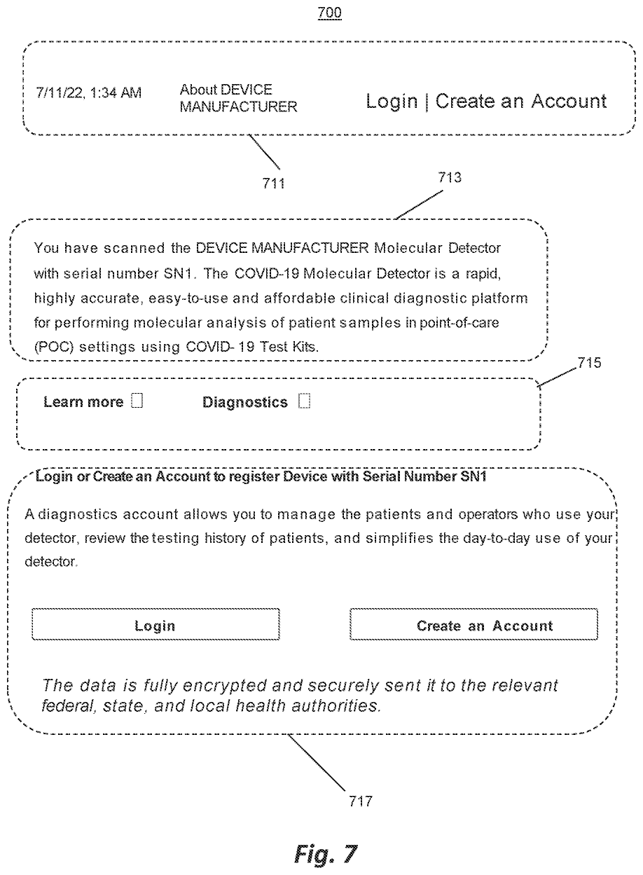

In various embodiments, upon scanning a static barcode (e.g. static barcode 190), using a suitable electronic device (e.g., a smartphone, a laptop, and the like) a test service provider (or a user) may be directed to a webpage based on the URL obtained from static barcode 190. An example URL constructed from static barcode 190 associated with diagnostic testing device 100 may be, for example, HTTPS://XX.XX/B00001Q:C1PCUBD$O9B6E126 which may direct a test service provider (or a user) to a website associated with the device 100 for the user to login, create an account, and/or provide personal information. In this way, test result data can be associated with (and later retrieved by) a user associated with the account and/or personal information. In an example embodiment, the website may display a device serial number based on the information obtained from static barcode 190. In some cases, the website may display the device serial number upon user logging into the website. Further, the website may display any other information associated with a device manufacturer (e.g., a device manufacturer name). In some cases, the website may display various links that may be accessed by the user. An example website 700, is shown in FIG. 7, including a login header 711, a message 713 that a QR code for the device was scanned, section 715 with possible links, and a region 717 for login, which may include any suitable message (e.g., a message that the data is encoded, and that the data may be sent to the government agencies, such as local state authorities). The static barcode 190 may allow the user to verify physical presence at the location of a device and verify physical possession of a device with a particular serial number without first having to run a test. This static QR code may facilitate management of ownership of devices in a detector portal website.

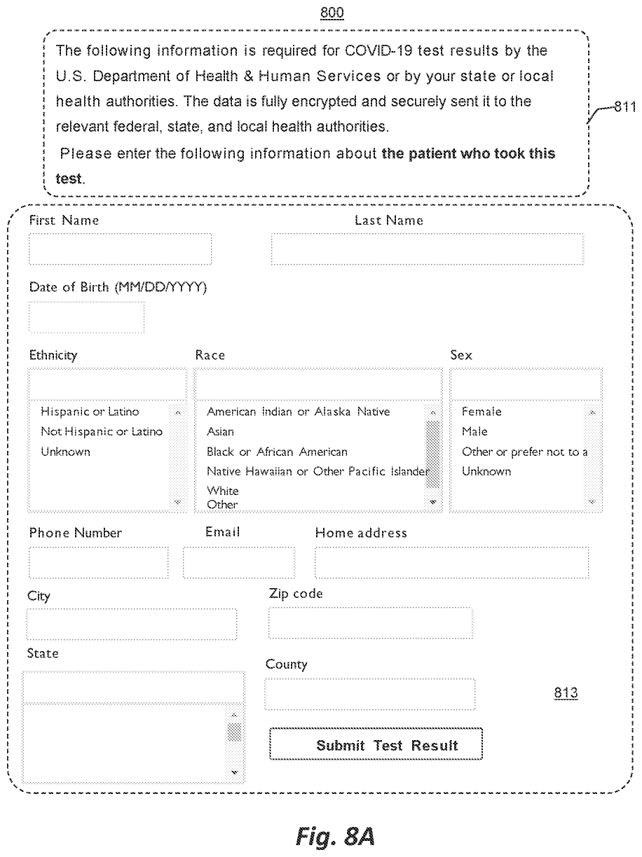

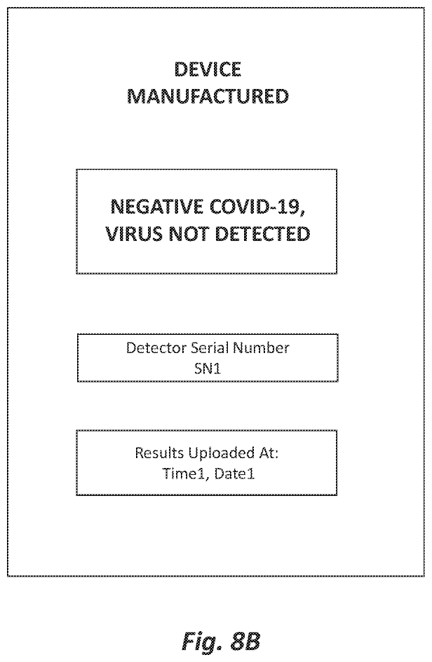

An example URL constructed from a dynamically generated barcode associated with device 100 (e.g., dynamic QR code 180, as show in FIG. 1) may be HTTPS://XX.XX/A008D1M::E583CTOKZ46J7LJ3R7.+Q1$D*3F8LTQ/QQZNLM+U. “HTTPS://XX.XX” can direct the device that scanned the URL to a website 800, as shown in FIG. 8A.

The payload, “A008D1M::E583CTOKZ46J7LJ3R7.+Q1$D*3F8LTQ/QQZNLM+U” may contain encoded information associated with the device, the test results, test operation, and/or personal information as described herein. In some instances, when the test service provider has access to user-related information (for example, when the test service provider is the user) the test service provider or the user may login to an associated account via website 800, enter user-related information at website 800 or create an account for the user. Website 800 may include a text message 811 informing the test service provider/user that the information may be encrypted and collected by relevant federal, state, and local health authorities. Further, the website 800 may include user information 813 such as first and last name of the user, data of birth of the user, ethnicity, race, and sex of the user, as well as the email, the address, and the phone number of the user. Upon filling user-related information, the test service provider may submit the test via a test submitting button (or any suitable test submitting graphical user element). Upon submitting the test result, the test result may be reported to the government authorities, and/or may be stored in a database associated with the user. It should be noted that user-related information (e.g., user identifying information such as the user name, the email, the address, and the phone number) may be removed when submitting the test-related information to the relevant federal, state, and local health authorities. In some instances, the website 800 may automatically pass test-related information to the relevant health authorities regardless of whether the personal information is submitted (e.g., when required by law.) In some embodiments, website 800 is configured to display test results as shown for example in FIG. 8B. The test results may include name of the device manufacturer, outcome of the test (e.g., NEGATIVE COVID-19), a serial number of the device 100 (e.g., serial number SN1) and time and date (e.g., Time1 and Data1) at which the test results were uploaded to the server.

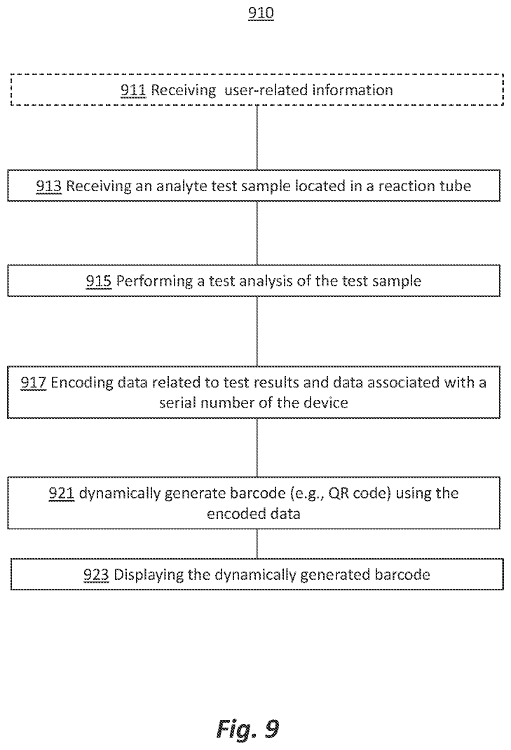

FIG. 9 shows an example process 910 for performing a diagnostic test and for displaying the diagnostic test information for submission to a server. Steps of process 910 may be performed by a device 100. At optional step 911, process 910 includes receiving user-related information from a user (e.g., the user-related information may be entered via an interface of the device 100). For instance, the user-related information may be any suitable unique user identification such as an account number of the user that can be scanned or entered manually into device 100. In one implementation, a sample may be taken from a patient at a medical facility by a medical service provider. For example, the swab sample from a patient, may be placed in a reaction tube by the medical service provider.

At step 913, device 100 is configured to receive a test sample in a reaction tube. The test sample may be any suitable fluid from a user, for example, the test sample may be extracted from a nasal swab, throat swab, and the like obtained from a user. At step 915, device 100 is configured to perform a test analysis using any suitable methods as previously referenced, and at step 917, device 100 is configured to encode data related to test results and data associated with a serial number of the device. At step 921, device 100 may be configured to dynamically generate a barcode based on the encoded data, and at step 923 device 100 is configured to display the dynamically generated barcode on a display associated with device 100.

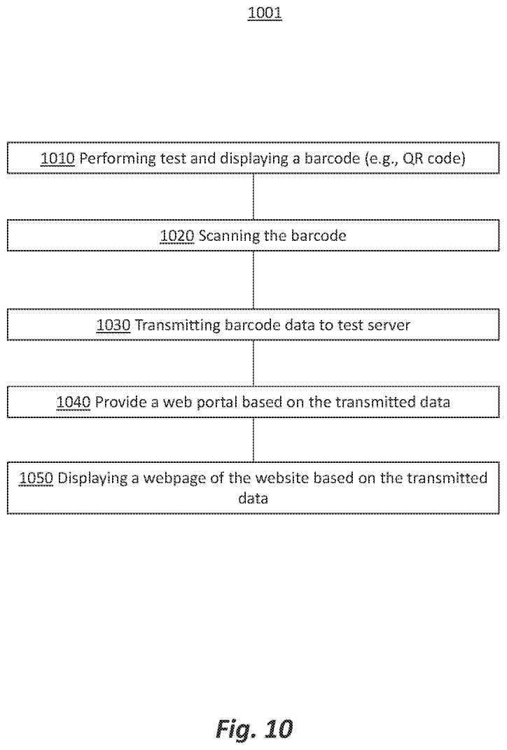

FIG. 10 shows an example process 1001 for performing a test and displaying a web page associated with the results of the test. In various embodiments, steps of process 1001 may be performed by a system of devices that may include a testing device, a scanning device and a server for serving a webpage associated with the results of the test. In an example embodiment, step 1010 includes performing a diagnostic test and displaying a dynamically generated barcode (e.g., a matrix code such as a QR code may be displayed on a display of a testing device). Step 1010 may be the same as process 910 (e.g., step 1010 may include steps 911-923 of process 910). In various embodiments, step 1010 is performed by a testing device (e.g., device 100). Further, process 1001 includes step 1020 of scanning an image of the dynamically generated barcode displayed by device 100 using any suitable scanning device (e.g., using a camera of a smartphone, or any other suitable device). In an example embodiment, the smartphone may be configured to include an application for scanning barcodes. At step 1030, the application for scanning barcodes may be configured to transmit barcode data encoded within the barcode to a test server. Upon receiving the barcode data, at step 1040, the test server is configured to provide a web portal based on the transmitted data (e.g., a web portal associated with the test service provider). In various cases, when providing the web portal based on the transmitted data, the transmitted data may be decoded. Further, after completion of step 1040, at step 1050, the application for scanning barcodes may be configured to display a webpage of a website in a web browser, the website or webpage may be displayed based on the transmitted data. In various embodiments, data within QR code may represent a URL having various parameters associated with information related to the test, as discussed above (e.g., URL HTTPS://XX.XX/A008D1M::E583CTOKZ46J7LJ3R7.+Q1$D*3F8LTQ/QQZNLM+U as discussed above).

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of the embodiments as discussed above.

For example, although device 100 described herein generally relate to FLOS-LAMP analysis and is particularly well suited to SARS-CoV-2 detection, it should be understood that device 100 can be applied to many other analyte that can be selectively amplified or concentrated through, for example, LAMP, polymerase chain reaction (PCR), chemical synthesis, electrochemistry, bioproduction, chromatography, electrophoresis, isoelectric focusing, gravimetric separation, etc. Although embodiments described herein generally describe fluorescent signals that are associated with or can be correlated to a quantity or concentration of an analyte, analytes can be detected by any suitable means such as, for example a pH-driven colorimetric signal from Real-Time Loop-Mediated Isothermal Amplification (RT-LAMP), or any other suitable colorimetric, electric, electro-chemical, optical absorbance, etc. signal.

In particular embodiments, device 100 may be used to determine the presence or absence of an analyte by detecting and/or measuring a signal generated via isothermal nucleic acid amplification. Isothermal amplification of nucleic acids is an alternative to polymerase chain reaction (PCR). The advantage of these methods is that the nucleic acids amplification can be carried out at constant temperature, unlike PCR, which requires cyclic temperature changes. In certain embodiments, the isothermal nucleic acid amplification is performed using, e.g., loop mediated isothermal amplification (LAMP), nucleic acid sequence based amplification (NASBA), Helicase dependent amplification (HDA), Exponential amplification reaction of nucleic acids (EXPAR), Strand displacement amplification (SDA), Recombinase polymerase amplification (RPA), rolling circle amplification (RCA), e.g., as described in O. L. Bodulev1 and I. Yu. Sakharov, Biochemistry (Moscow), 2020, Vol. 85, No. 2, pp. 147166 and references cited therein, which is incorporated by reference herein in its entirety.

In some embodiments, a test sample for the diagnostic test is a biological sample obtained from a subject diagnosed with an infection or considered to be at risk of having or developing the infection. In other embodiments, the sample is a food product or beverage product. In some embodiments, the sample is obtained from a surface, e.g., a food preparation surface, a food or beverage package surface, or a surface in a home, rental home, or hotel, such as but not limited to a kitchen counter surface, a bathroom counter surface, a toilet, shower, or bathtub surface, or a table or dresser surface.

In certain embodiments, the analyte is a virus or component thereof. In some embodiments, the test sample is a biological sample obtained from a subject diagnosed with or suspected of being or at risk of being infected with the virus. For example, the test sample may be a nose swab, a throat swab, and the like. In particular embodiments, the virus is a norovirus, rotavirus, adenovirus, astrovirus, influenza virus, coronavirus, parainfluenza virus, respiratory syncytial virus, human immunodeficiency virus (HIV), human T lymphotropic virus (HTLV), rhinovirus, hepatitis A virus, hepatitis B virus, Epstein Barr virus, or West Nile virus. In particular embodiments, the virus is SARS-CoV-2.

In addition, some methods described herein describe terminating a sample run when a target or control indicates (optionally, after a waiting period). It should be understood, however, that in other embodiments, the sample can be run (e.g., the target analyte can be selectively amplified) to a maximum duration (e.g., 60 minutes, 90 minutes, etc.). In such an embodiment, an indication of a positive result can be sent if the target has indicated in that time period (optionally accepting indications during an excluded initial period). An indication of a negative result can be sent if the control has indicated in the time period. In other scenarios, a signal indicating that the test has failed or is indeterminate can be sent.

Where methods and/or events described above indicate certain events and/or procedures occurring in a certain order, the ordering of certain events and/or procedures may be modified. Additionally, certain events and/or procedures may be performed concurrently in a parallel process when possible, as well as performed sequentially as described above.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, and the like, that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium (e.g., a non-transitory computer-readable storage medium) such as a hard disk, optical disk, removable storage media, solid-state memory, RAM, and the like. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a system, and/or a computer program product.

### APPENDIX

1. A device comprising:
   a well configured to receive a reaction tube containing a test sample and an analyte;
   a light emitting source configured to emit an excitation light at a wavelength to illuminate the analyte in the reaction tube;
   an optical detector configured to receive an emission light in response to the analyte being illuminated by the excitation light;
   a processor operably coupled to the light emitting source and the optical detector, wherein the processor is configured to:
      analyze the emission light to determine results of a diagnostic test for determining if a patient has a viral or a bacterial infection,
      encode data related to the results of the diagnostic test, and
      form a dynamic QR code using the encoded data, wherein the dynamic QR code includes the data associated with the results of the diagnostic test and is configured to be scanned using an electronic device; and
   a display configured to display the dynamic QR code.
2. The device of claim 1, further comprising: a static QR code associated with the device, wherein the processor receives the static QR code and encodes the data from both the results of the diagnostic test and data associated with the static QR code.
3. The device of claim 1, further comprising: a receiver configured to receive wirelessly transmitted test-related data from a chip coupled to the reaction tube.
4. The device of claim 3, wherein the test-related data includes a type of test performed.
6. The device of claim 3, further comprising:
   a hinged cover configured to be movable to cover a top portion of the reaction tube,
   the receiver located within the hinged cover.
7. The device of claim 1, wherein the dynamic QR code includes an encoding character sequence that includes one or more encoding characters used for encoding the data related to the results of the diagnostic test.
8. The device of claim 7, wherein the encoding characters used for encoding information in the dynamic QR code is arranged in a bit-packed data structure (BPDS).
9. The device of claim 8, wherein the BPDS includes a timestamp including a length of time needed for detecting a particular one of outcomes such as one or more of the following: positive, negative, malfunction, inconclusive, or canceled.
10. The device of claim 1, wherein the light emitting source includes one or more light emitting diodes.
11. The device of claim 1, wherein the device is a Fluorescence of Loop Primer Upon Self Dequenching Loop-Mediated Isothermal Amplification (FLOS-LAMP) device operable to amplify the analyte and measure a signal associated with a quantity of the analyte.
12. The device of claim 1, wherein the test sample is a biological sample and the biological sample is selected from the group consisting of: serum, blood, salivary secretions, lacrimal secretions, respiratory secretions, nasal fluid, a mucous sample, and intestinal secretions.
13. The device of claim 1, wherein the processor is configured to communicate the results of the diagnostic test to a patient in the form of one of the dynamic QR code or the data from the results of the diagnostic test.
14. The device of claim 1, wherein the processor is configured to communicate the results of the diagnostic test to a medical professional in the form of one of the dynamic QR code or the data from the results of the diagnostic test.
15. The device of claim 1, wherein the processor is configured to communicate the results of the diagnostic test to an agency for analyzing pathogen transmission within a population in the form of one of the dynamic QR code or the data from the results of the diagnostic test.
16. A kit for use with the device of claim 1 comprising, a set of instructions, a nasal swab, a buffer tube, a transfer pipette, and the reaction tube.
17. A method, comprising:
   receiving a test sample located in a reaction tube containing the test sample and an analyte;
   performing a test analysis of the test sample and the analyte, wherein the test analysis includes results of a diagnostic test for determining if a patient has a viral or bacterial infection;
   encoding data relating to the results of the diagnostic test and other data associated with a serial number of the device;
   forming a dynamic QR code using the encoded data; and
   displaying the dynamic QR code.
18. The method of claim 17, further comprising:
   scanning, by an application for scanning QR codes on a scanning device, the dynamic QR code;
   transmitting, by the application and the scanning device, the data relating to the results of the diagnostic test to a test server;
   creating a website, by the test server, wherein the website includes the data relating to the results of the diagnostic test; and
   displaying the website, by the application and the scanning device, based on the data relating to the results of the diagnostic test.
19. The method of claim 17, further comprising:
   emitting, by a light emitting source, an excitation light to illuminate the analyte in the reaction tube;
   receiving, by an optical detector, an emission light in response to the analyte being illuminated by the excitation light; and
   determining, by a processor operably coupled to the light emitting source and the optical detector, at least one of a quantity or a concentration of the analyte by causing the analyte to be illuminated by the light emitting source and the emission light to be received and processed.
20. A system, comprising:
   a device including:
      a housing defining a well, configured to receive at least one reaction tube from a plurality of reaction tubes, wherein the at least one reaction tube contains a test sample and an analyte;
      a receiver configured to receive any one of a plurality of near-field communication signals from the at least one reaction tube, each near-field communication signal from the plurality of near-field communication signals including a plurality of parameters for performing an assay on the test sample contained in the at least one reaction tube;
      a light emitting source configured to emit an excitation light to illuminate the sample in the reaction tube;
      an optical detector configured to receive an emission signal in response to the sample being illuminated by the excitation light;
      a processor operably coupled to the receiver, the light emitting source, and the optical detector, wherein the processor is configured to:
         analyze the emission light to determine results of a diagnostic test for determining if a patient has a viral or bacterial infection,
         encode data related to the results of the diagnostic test, and
         form a dynamic QR code using the encoded data, wherein the dynamic QR code includes the data associated with the results of the diagnostic test and is configured to be scanned using an electronic device; and
      a display configured to display the dynamic QR code;
      and
   the plurality of reaction tubes, each reaction tube from the plurality of reaction tubes configured to be inserted into the well,
      a first reaction tube from the plurality of reaction tubes including a first near-field communication chip configured to transmit a first signal including a first plurality of parameters for performing a first assay,
      a second reaction tube from the plurality of reaction tubes including a second near-field communication chip configured to transmit a second signal including a second plurality of parameters for performing the second assay, at least one of the second plurality of parameters being different from at least one of the first plurality of parameters such that the second assay is different from the first assay.

What is claimed is:

1. A method for visually indicating the results of a diagnostic test comprising:
performing, using a diagnostic testing device, a diagnostic test at least by analyzing, using a light emitting source and an optical detector, emission light from an analyte combined with a test sample in a reaction tube residing within a well of the detector;
obtaining, based on the analyzing, one or more test results indicating whether a patient that provided the test sample has a viral or bacterial infection;
generating, based on a size of a display screen of the diagnostic testing device, a barcode configured to cause, based on being scanned by a scanning device of a computing device, the computing device to automatically send, to a remote data store, the one or more test results obtained, wherein the generating the barcode comprises:
generating a bit-packed data structure (BPDS) comprising a bit sequence indicating the one or more test results obtained;
obtaining an encoded BPDS by encoding, based on a storage capacity of the barcode, the BDPS;
generating, based on the storage capacity of the barcode, a URL comprising the encoded BPDS; and
encoding, in the barcode, the URL generated; and
presenting, on the display screen of the diagnostic testing device.

2. The method of claim 1, wherein:
a core of the BPDS comprises the bit sequence indicating the one or more test results obtained;
the generating the barcode further comprises:
generating, based on a key stored in firmware of the diagnostic testing device and based on a serial number of the diagnostic testing device, a keyed-hash message authentication code (HMAC); and
obtaining a truncated HMAC by truncating the HMAC; and
the generating the BPDS comprises appending the truncated HMAC to a core of the BPDS.

3. The method of claim 1, wherein the BPDS further comprises at least one of:
a bit sequence indicating a format of a core of the BPDS;
a bit sequence indicating a product type associated with the diagnostic testing device;
a bit sequence indicating a serial number of the diagnostic testing device;
a bit sequence indicating a test run number of the diagnostic test;
a bit sequence indicating a type of the diagnostic test;
a bit sequence indicating a statistic associated with previous diagnostic tests performed by the diagnostic testing device wherein the statistic indicates one of:
a quantity of test results indicating a positive infection;
a quantity of test results indicating a negative infection;
a quantity of test results determined to be inconclusive;
a quantity of diagnostic tests during which a malfunction occurred; or
a quantity of diagnostic tests that were canceled;
a time that elapsed between a start of the diagnostic test an detection of a target infection;
a time that elapsed between a start of the diagnostic test and detection of a control signal; or
an error code.

4. The method of claim 1, wherein the barcode comprises at least one of:
a two-dimensional (2D) barcode;
a matrix barcode; or
a Quick Response (QR) code.

5. The method of claim 1, wherein the encoding the BPDS comprises encoding the BPDS using an encoding scheme that minimizes a size of the encoded BPDS.

6. The method of claim 1, wherein generating the URL comprises using a fully qualified domain name that minimizes a size of the URL.

7. The method of claim 1, wherein the diagnostic testing device comprises no wired or wireless network interface used to provide the one or more test results obtained.

8. A diagnostic testing device configured to visually indicate the results of a diagnostic test comprising:
a well configured to receive a reaction tube containing an analyte and a test sample provided by a patient;
a light source configured to illuminate the analyte in the reaction tube with excitation light;
an optical detector configured to receive emission light from the analyte based on the analyte being illuminated with the excitation light;
a display screen;
a processor in signal communication with the display screen, the light source, and the optical detector; and
memory storing instructions that, when executed by the processor, cause the diagnostic testing device to perform the method of any one of claims 1-7.

9. A non-transitory computer-readable medium storing instructions that, when executed by a processor of a diagnostic testing device, cause the diagnostic testing device to visually indicate the results of a diagnostic test at least by performing the method of any one of claims 1-7.
